(19) United States
(12) Patent Application Publication
(10) Pub. No.: US 2005/0137545 A1
(43) Pub. Date: Jun. 23, 2005

(54) Method of forming an absorbent body

(75) Inventor: Lars Fingal, Goteborg (SE)

Correspondence Address: YOUNG & THOMPSON, 745 SOUTH 23RD STREET, 2ND FLOOR, ARLINGTON, VA, 22202, US

(73) Assignee: SCA HYGIENE PRODUCTS AB, GOTEBORG (SE)

(21) Appl. No.: 11/017,692

(22) Filed: Dec. 22, 2004

Related U.S. Application Data
(60) Provisional application No. 60/530,903, filed on Dec. 22, 2003.

(51) Int. Cl. A61F 13/15; A61F 13/20

(52) U.S. Cl. 604/367

(57) ABSTRACT

A method of forming an absorbent body having coarse stripes in the machine direction by wet-laying fibres onto a liquid permeable web running in the machine direction. According to the invention, the flow of liquid through the liquid permeable web is blocked in at least one elongate region essentially directed in the machine direction, thereby a wet-laid fibre body is obtained. A fibrous absorbent body manufactured by the method is also disclosed.

### CROSS REFERENCE TO RELATED APPLICATION

This application claims the 35 USC 119(e) benefit of prior U.S. Provisional application Ser. No. 60/530,903 filed on 22 Dec. 2003, the entirety of which is expressly incorporated by reference.

### TECHNICAL FIELD

The present invention relates to a method of forming an absorbent body having coarse stripes in the machine direction by wet-laying fibres onto a liquid permeable web running in the machine direction. The invention also relates to an absorbent body produced thereby.

### BACKGROUND OF THE INVENTION

It is known to form patterns in wet-laid absorbent bodies by embossing or by the use of a pattern in the wire, onto which the fibres are laid. Such methods are suitable to form fine patterns in absorbent bodies but not appropriate for forming coarse stripes in an absorbent body. For certain applications, such as a scouring-cloths, swabs or industrial wipes, it is desirable to have a pattern of coarse stripes in an absorbent body.

The objective of the present invention is to provide a simple and straightforward way of producing an absorbent body having a pattern of coarse stripes.

### SUMMARY OF THE INVENTION

This objective is accomplished by a method of forming an absorbent body having coarse stripes in the machine direction by wet-laying fibres onto a liquid permeable web running in the machine direction, characterised by blocking the flow of liquid through the liquid permeable web in at least one elongate region essentially directed in the machine direction, thereby obtaining a wet-laid fibre body. In such a method most of the slurry of liquid and fibres will pass outside the blocked region and a very little amount of fibres will be laid down on the blocked region in relation to the amount be laid onto the web outside the blocked region.

In a first preferred embodiment a suction box is blocked in at least one elongate region essentially directed in the machine direction, the suction box being disposed below a chamber delivering fibrous slurry onto a liquid permeable web.

In a second preferred embodiment at least one elongate region essentially directed in the machine direction is blocked in the liquid permeable web, onto which fibrous slurry is laid.

In a first alternative of the second embodiment said liquid permeable web is a first web of nonwoven material having at least one liquid impermeable longitudinal region essentially directed in the machine direction. A second web of nonwoven material can be laid onto said first web of nonwoven material after said fibre body has been obtained.

In a second alternative of the second embodiment said liquid permeable web is a formation belt having at least one liquid impermeable longitudinal region essentially directed in the machine direction.

The wet-laid absorbent bodies and possible webs of nonwoven material are preferably subjected to hydroentangling.

The invention also relates to a fibrous absorbent body, characterised by a pattern of longitudinal stripes being formed therein, the distance between adjacent stripes being less than 2 cm.

In a preferred embodiment the absorbent body is enclosed between two layers of nonwoven material, the layers of nonwoven material being attached to the absorbent body and each other by bonds produced by entangling, and in that one of the layers of nonwoven material has a pattern of longitudinal liquid impermeable regions, each region being located between adjacent stripes in the pattern of stripes formed in the absorbent body.

### BRIEF DESCRIPTION OF THE DRAWINGS

The invention will now be described with reference to the enclosed figures, of which;

FIG. 1 shows a perspective view of an absorbent body according to a preferred embodiment of the present invention,

FIG. 2 shows a sectional view along line II-II in FIG. 1, and

FIGS. 3 and 4 illustrate schematically a side view and a plan view, respectively of an apparatus for manufacturing an absorbent body according to a preferred embodiment of the invention.

### DESCRIPTION OF THE PREFERRED EMBODIMENTS

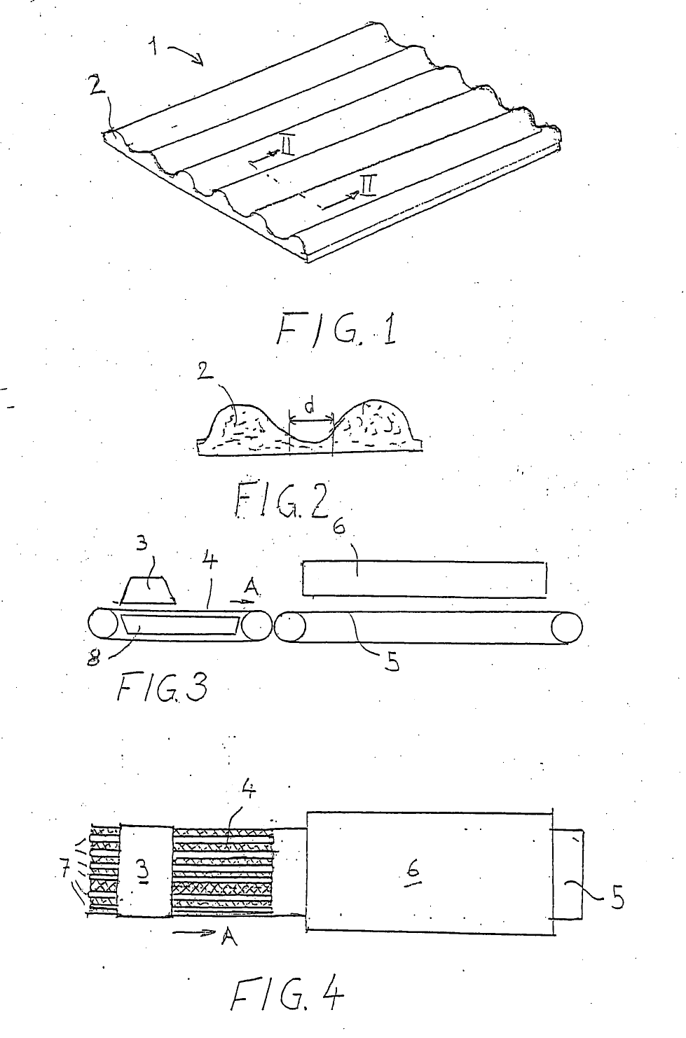

In FIGS. 1 and 2 a first embodiment of an absorbent body 1 having a pattern of longitudinal stripes 2 is shown. The absorbent body preferably consists of pulp fibres with or without synthetic staple length fibres being mixed therein. The fibres are bonded to each other by bonds being formed by entangling. The bonds thereby consist of mechanical bonds and hydrogen bonds between pulp fibres, the mechanical bonds being constituted of fibres entwined or entwisted with or around each other. The distance d between adjacent stripes 2 is less than about 2 cm and more than about 0.5 cm.

Such an absorbent body with such coarse stripes is suitable to use as scouring-cloth, swab or industrial wipe.

In FIGS. 3 and 4 an apparatus for manufacturing the absorbent body shown in FIGS. 1 and 2 is schematically shown.

The apparatus comprises a conventional wet-laying unit 3 laying fibrous slurry onto an endless wire 4 or a formation belt. The fibres laid down on wire 4 is then transferred to a conveyor 5 and passes under a conventional hydroentangling unit 6, in which several rows of nozzles for water jets are present. A suction box 8 is disposed under the wire 4 as is schematically shown in FIG. 3 and a suction box (not shown) is also present under the hydroentangling unit 6. The apparatus also contains a water circuit for taking care of the water drained from the wire 4 and also a water circuit for the water in the hydroentangling unit, said circuits not being shown in the figures.

According to the invention the wire 4 comprises a row of elongate liquid tight blocking elements 7, for example plastic strips, directed in the machine direction A and extending around the periphery of the wire. Otherwise, the apparatus is of conventional construction well known to the skilled man.

When the flow of fibrous slurry in the wet-laying unit 3 encounters the wire, the liquid impermeable regions 7 totally block the flow of fibrous slurry and the slurry will flow through the liquid permeable areas between the regions 7. The water in the slurry will flow through the wire and the fibres therein will be captured by the wire. Thereby, almost all of the fibres in the slurry will accumulate on the liquid permeable regions thereof. Above the regions 7 the flow of fibrous slurry will have a horizontal component in order to reach the liquid permeable regions of the wire. More fibres will be accumulated in the middle of the permeable regions between impermeable regions than along the longitudinal edges of the permeable regions and the formed stripes will have thus have inclined edges. Due to the pressure difference between the upper side and lower side of the wire, a small horizontal flow of slurry from the impermeable regions towards the longitudinal edges thereof will also occur and some fibres will therefore accumulate on the impermeable regions of the wire, the thickness of this fibre layer increasing from the middle of the impermeable region in a direction towards the longitudinal edges thereof. In order for the impermeable regions to have the described blocking effect their width should be at least 0.5 cm.

As stated above, most of the fibres in the slurry will accumulate along the permeable regions of the wire and there is therefore a risk that the wet-laid absorbent body will have a too small amount of fibres accumulated between adjacent stripes for holding the body together. In order to ensure that the regions between the stripes have sufficient strength, the distance between adjacent stripes should preferably not exceed 2 cm.

If it is desired to increase the thickness of the regions between adjacent stripes in an absorbent body produced in accordance with the method described above, this can be done by making the elements 7 of a material that will not totally block the flow of water therethrough, such as a textile material with a small liquid permeability compared to the permeability of the other regions of the wire.

An alternative way of blocking the flow of liquid through a wire or the like is to block the suction box instead of the wire. In such an alternative, there will always be some flow of water through the parts of the wire located immediately above the blocked parts of the suction box but most of the flow of slurry will be directed towards the non-blocked parts of the suction box. Also in this case the blocking can be made by elongate blocking elements disposed on the suction box, the amount of blocking can be varied by varying the liquid permeability of the blocking elements.

A further alternative to manufacture an absorbent body according to FIGS. 1 and 2 is to lay the fibrous slurry onto a substrate having a pattern of blocking regions directed in the machine direction. The substrate can be a nonwoven material, for example a spunlaid web. Also in this case the blocking can be varied by varying the permeability of those regions. The wet-laid body with its pattern of stripes can be transferred to the entangling unit together with the substrate or not. If the substrate is to be separated from the wet-laid body before entering the entangling unit, the substrate is suitably attached to the periphery of the endless wire and follows the movements of the wire.

By using a nonwoven web as substrate, a wet mopping material consisting of a striped core enclosed between two nonwoven webs can be obtained in a one-step process. The only modification of the apparatus described in FIGS. 3 and 4 for such an application is to provide means for laying a second nonwoven web preferably lacking a pattern of liquid impermeable regions, onto the composite of a lower nonwoven web and wet-laid absorbent body before entering the entangling unit. By the entangling the nonwoven webs will be attached to each other and to the fibrous absorbent body.

The fibres to be used in the present invention can be natural fibres and staple fibres. The natural fibres are preferably wood pulp fibres but any type of cellulosic fibres is possible to use, such as grass or straw. Both softwood fibres and hardwood fibres are suitable. It is also possible to use regenerated cellulose fibres, such as rayon, lyocell. The staple fibers can be synthetic fibres made from polypropylene or polyesters and copolymers of these materials can of course also be used. Other polymers, such as polyethylene, polyamides or polyactides, can also be used.

The entangled web material leaving the entangling unit is preferably subjected to a drying process by any suitable means and is rolled up on a storage roll or cut into separate absorbent bodies or wet-mopping bodies and packaged.

The described embodiments can of course be modified within the scope of the invention. For example, the stripes formed and the regions without or with a low liquid permeability need not be parallel with the machine direction but can deviate a small amount from the machine direction. The stripes formed and said regions need further not be totally straight but curved forms, such as sine curve, can also be used. Moreover, the distances between the stripes can be varied. The wet-laying of the fibres can include a foam-forming step and more than one hydroentangling units can be used. The scope of invention shall therefore only be restricted by the content of the enclosed patent claims.

1. A method of forming an absorbent body having coarse stripes in a machine direction which comprises:
wet-laying fibres onto a liquid permeable web running in the machine direction; and
blocking the flow of liquid through the liquid permeable web in at least one elongate region essentially directed in the machine direction, thereby obtaining a wet-laid fibre body.

2. The method according to claim 1, further comprising blocking a suction box in at least one elongate region essentially directed in the machine direction, the suction box being disposed below a chamber delivering fibrous slurry onto the liquid permeable web.

3. The method according to claim 1, further comprising blocking at least one elongate region essentially directed in the machine direction in the liquid permeable web, onto which fibrous slurry is laid.

4. The method according to claim 3, wherein said liquid permeable web is a first web of nonwoven material having at least one liquid impermeable longitudinal region essentially directed in the machine direction.

5. The method according to claim 3, wherein said liquid permeable web is a formation belt having at least one liquid impermeable longitudinal region essentially directed in the machine direction.

6. The method according to claim 1, further comprising hydroentangling said wet-laid fibre body.

7. The method according to claim 5, further comprising laying a second web of nonwoven material onto said first web of nonwoven material after said fibre body has been obtained, and subjecting the resulting composite to hydroentangling.

8. A fibrous absorbent body comprising a pattern of longitudinal stripes formed therein, the distance between adjacent stripes being less than about 2 cm and more than about 0.5 cm.

9. The absorbent body according to claim 8, wherein the absorbent body is enclosed between two layers of nonwoven material, the layers of nonwoven material being attached to the absorbent body and each other by bonds produced by entangling; one of the layers of nonwoven material having a pattern of longitudinal liquid impermeable regions, each region being located between adjacent stripes in the pattern of stripes formed in the absorbent body.
